(19) United States
(12) Patent Application Publication
(10) Pub. No.: US 2023/0218299 A1
(43) Pub. Date: Jul. 13, 2023

(54) LEAD METHOD OF SUTURING ACUTELY RUPTURED ACHILLES TENDON AND DEVICE USED IN THE LEAD METHOD

(71) Applicant: ZHENGZHOU ORTHOPAEDICS HOSPITAL, Zhengzhou (CN)

(72) Inventor: Changsong CAO, Zhengzhou (CN)

(73) Assignee: ZHENGZHOU ORTHOPAEDICS HOSPITAL, Zhengzhou (CN)

(21) Appl. No.: 17/725,839

(22) Filed: Apr. 21, 2022

(30) Foreign Application Priority Data
Jan. 13, 2022 (CN) 202210035231.0

(51) Int. Cl. A61B 17/11; A61B 17/04

(52) U.S. Cl. CPC A61B 17/1146; A61B 17/0482; A61B 2017/1125

(57) ABSTRACT

Disclosed are a lead method of suturing an acutely ruptured Achilles tendon and a device used in it. The suturing device includes a suture line, guide lines, a clamping instrument and suture needles. As performing suturing, the clamping instrument is inserted into the fascia sheath of Achilles tendon to clamp Achilles tendon, the suture line and the guide lines are made to penetrate Achilles tendon and skin through the threading hole on the clamping instrument by the suture needle, wherein the suture line is located far away from Achilles tendon rupture; the guide lines are used to guide the suture line, so that both ends of the suture line can penetrate Achilles tendon in turn along the guide lines, and the suture line is tightened and knotted on the two ruptured Achilles tendons.

### CROSS-REFERENCE TO RELATED APPLICATIONS

This application claims priority to Chinese Patent Application No. 202210035231.0, filed on Jan. 13, 2022, the contents of which are hereby incorporated by reference.

### TECHNICAL FIELD

The application relates to the technical field of medical instruments, and in particular to a lead method for suturing acute Achilles tendon (tendo calcaneus) rupture and a device for its application.

### BACKGROUND

Acute Achilles tendon rupture is a common injury in labor work, military training and sports, where the common rupture is 2-6 cm up from the Achilles stop, and Achilles tendon rupture is relatively common among tendon ruptures. The recovery of Achilles tendon ruptures is often not ideal due to tendon adhesion, scar repairs, poor healing and infection. At present, in order to repair and connect the Achilles tendon, a long incision is required to reveal the normal part of the tendon and to strip the peritendinous tissue in order to fix the suture to the normal tendinous tissue, but this inevitably causes damage to the local blood circulation of the Achilles tendon, resulting in complications such as tendon adhesion, scar repair, poor healing and infection, etc.

In recent years, the method of treating acute Achilles tendon rupture with small percutaneous incisions emerges with similar efficacy to incisional sutures and a significantly lower complication rate. However, minimally invasive percutaneous sutures are not yet widely used in clinical practice due to complex and costly minimally invasive sutures; moreover, percutaneous minimally invasive sutures sometimes require multiple small secondary incisions and carry the risk of medically induced peroneal nerve injury. Furthermore, under the existing minimally invasive percutaneous suture method, the tension of the ruptured Achilles tendon after healing is not as strong as that of Achilles tendon treated by weaving suture, and there is still a risk of re-rupture of the healed Achilles tendon.

### SUMMARY

The purpose of the present application is to provide a lead method of suturing an acutely ruptured Achilles tendon and a device used in the lead method to solve problems existing in the prior art.

To achieve the above objectives, the present application provides the following technical scheme: a lead method of suturing an acutely ruptured Achilles tendon specifically including the following steps:

S1: positioning an Achilles tendon by inserting a clamping instrument into a fascia sheath of the Achilles tendon through a small skin incision to clamp the Achilles tendon;

S2: threading the Achilles tendon by using a suture needle to make a suture line and guide lines penetrate the Achilles tendon and skin from the threading hole on the clamping instrument, wherein the suture line is located far away from an Achilles tendon rupture, and the guide lines are located between the suture line and the Achilles tendon rupture;

S3: leading-out the suture line and the guide lines by withdrawing the clamping instrument, and leading the suture line and the guide lines out of the small skin incision by using the threading hole on the clamping instrument;

S4: fixing the suture line by guiding the suture line with the guide lines, so that both ends of the suture line sequentially penetrate through the Achilles tendon along the guide lines and suture the ruptured Achilles tendon; and

S5: connecting the Achilles tendon by tightening, knotting and fixing the suture line on two broken parts of the Achilles tendon.

Preferably, before positioning the Achilles tendon, the small skin incision is made at a broken site of the Achilles tendon, and the small skin incision is between 1.5 cm and 2.5 cm long.

Preferably, when the suture line is fixed, the suture line is inserted into coils on the guide lines, and the coils are used to limit the suture line; the coils on the guide lines move with the guide lines and pass through an Achilles tendon channel of the guide lines.

Preferably, an end of the suture line on a same side of the coils penetrates into the coils on the guide lines respectively, so that the suture line passes through the Achilles tendon channel of the guide lines.

Preferably, an end of the suture line on an opposite side of the coils is inserted into the coils on the guide lines respectively, so that the suture line passes through the Achilles tendon channel of the guide lines after bypassing the Achilles tendon.

Preferably, when connecting Achilles tendon, the suture line is tightened to make the two ruptured Achilles tendons in close contact, and a redundant suture line is cut off after the suture line is tied and fixed.

A device used in the lead method of suturing an acutely ruptured Achilles tendon comprises:
a suture line fixedly stitched on a broken Achilles tendon and used to connect the broken Achilles tendon;
guide lines used for guiding the suture line; each guide line being provided with a coil in a middle of the guide lines for limiting the suture line, so as to ensure that the suture line moves along with the guide lines and passes through an Achilles tendon channel of the guide lines;
a clamping instrument used for clamping and fixing the Achilles tendon; front ends of the clamping instrument being provided with threading holes, and the threading hole being used for positioning the suture line and the guide lines; and
suture needles, through which the suture line and the guide lines pass through the Achilles tendon and skin through the threading holes on the clamping instrument.

Preferably, the clamping instrument is clamping anastomosis forceps, and a buckle is arranged at a tail end of the clamping instrument; the buckle is used for fixing an opening and closing angle of the clamping instrument so as to clamp and fix the Achilles tendon.

Compared with the prior art, the application has the following technical effects:
1. the surgical incision is small, requiring only a skin incision of approximately 2.0 cm long with stripping of the peritendinous tissue and little damage to the blood supply system of the skin and Achilles tendon; the skin incision and Achilles tendon heal under normal blood supply, which greatly reduces the post-operative complications, including skin necrosis of the incision, exposure of the Achilles tendon, post-operative infection, peroneal nerve injury, Achilles tendon adhesion, scarring repair and poor healing;
2. the lead method is technically ingenious so that the suture line could follow the left and right movements of the guide lines, and the ruptured Achilles tendon is sutured through the Achilles tendon channel of the guide lines; Achilles tendon sutured in this method is firm and reliable, and the resistance to tension of the ruptured Achilles tendon is relatively strong after the completion of the Achilles tendon anastomosis, so that the Achilles tendon is unlikely to rupture again after the operation, and the functional recovery of the Achilles tendon is rather reliable;
3. the suture line, locating inside the tissues, does not compress the body and has little impact on the tissues, which ensures normal blood circulation in the lower leg and promote the healing of the Achilles tendon; and
4. the lead method offers easy operation, simple auxiliary tools, and it could be used widely as being less expensive.

### BRIEF DESCRIPTION OF THE DRAWINGS

In order to more clearly explain the embodiments of the present application or the technical solutions in the prior art, the drawings needed in the embodiments will be briefly introduced below. Obviously, the drawings in the following description are only some embodiments of the present application, and for ordinary technicians in the field, other drawings could be obtained according to these drawings without creative efforts.

FIG. 1 shows a schematic structural diagram of the device of the present application.

FIG. 2 shows a structural diagram of the guide lines of the present application.

FIG. 3 shows a flow chart illustrating the lead method of the present application.

FIG. 4 shows a processing of sewing using the sewing device in the Embodiment 1 of the present application.

FIG. 5 shows a processing of sewing using the sewing device in the Embodiment 2 of the present application.

### DETAILED DESCRIPTION OF THE EMBODIMENTS

Next the technical solutions in the embodiments of the present application will be clearly and completely described with reference to the drawings in the embodiments of the present application. Obviously the described embodiments are only part of the embodiments of the present application, not all of them. Based on the embodiments in the present application, all other embodiments obtained by ordinary technicians in the field without creative work are within the protection scope of the present application.

In order to make the above-mentioned objectives, features and advantages of the present application more obvious and easier to understand, the present application will be described in further detail below with reference to the drawings and detailed description.

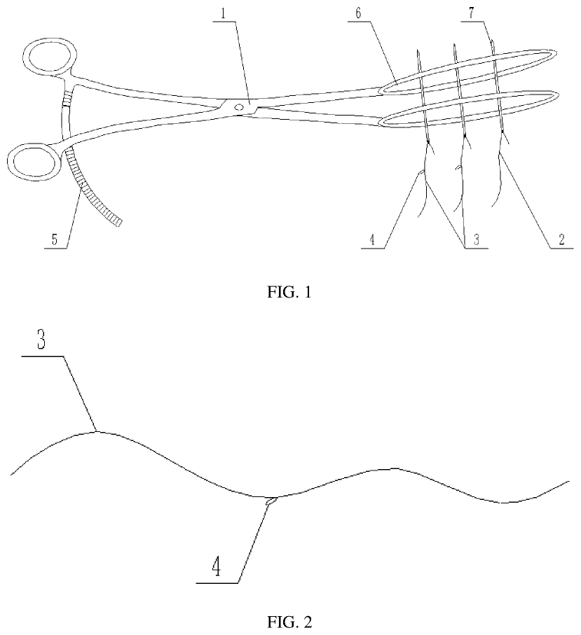

The present application provides a device used in the lead method of suturing an acutely ruptured Achilles tendon as shown in FIGS. 1-2, including:
a suture line 2, which is fixedly sewed on the broken Achilles tendon, and is used to connect the broken Achilles tendon;
guide lines 3, which is used to guide the suture line 2; each guide line is provided with a coil 4 formed by knotting the guide lines 3 itself, where the coil 4 is used to limit the suture line 2 to ensure that the suture line 2 moves along with the guide lines 3;
a clamping instrument 1, it is a clamping anastomosis forceps provided with a buckle 5 at the tail end of the clamping anastomosis forceps, and the buckle 5 is used for fixing the opening and closing angle of the clamping instrument 1 so as to clamp and fix the Achilles tendon; the front end of the clamping instrument 1 is provided with threading holes 6 for positioning the suture line 2 and the guide lines 3;
suture needles 7, where the suture line 2 and the guide lines 3 all pass through Achilles tendon and skin through the threading hole 6 of the clamping instrument 1 by the suture needle 7.

### Embodiment 1

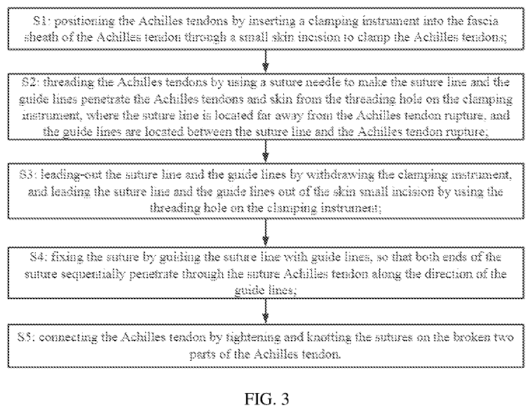

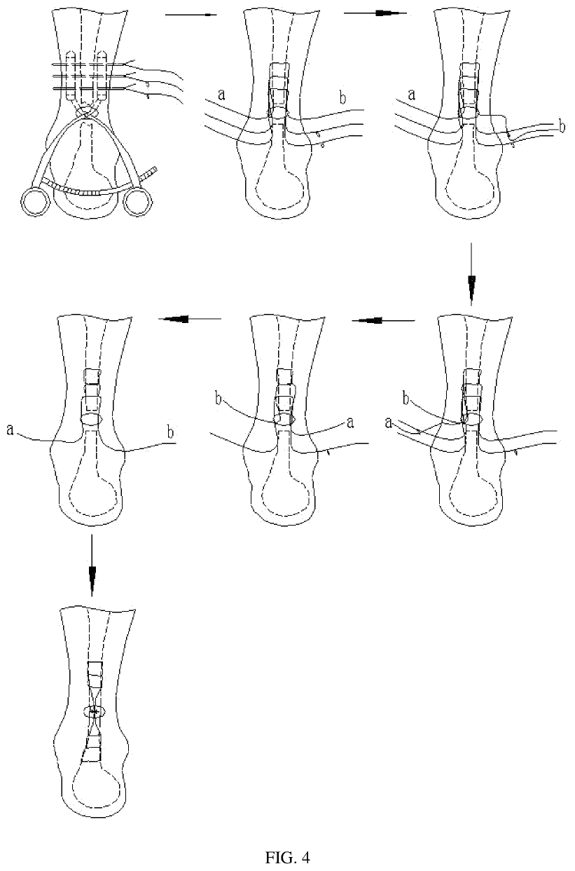

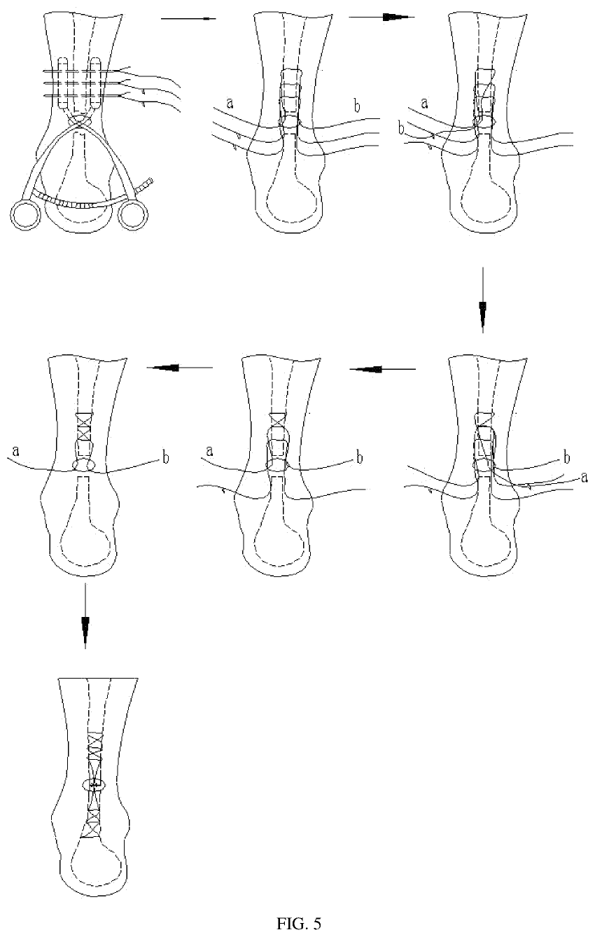

A lead method of suturing an acutely ruptured Achilles tendon is shown in FIGS. 3 and 4, which specifically include the following steps:
S1: positioning an Achilles tendon by inserting a clamping instrument 1 into the fascia sheath of the Achilles tendon through a small skin incision to clamp the Achilles tendon;
S2: threading the Achilles tendon by using suture needle 7 to make suture line 2 and guide lines 3 penetrate the Achilles tendon and skin from threading hole 6 on clamping instrument 1, where suture line 2 is located far away from Achilles tendon rupture, and guide lines 3 are located between suture line 2 and Achilles tendon rupture;
S3: leading out the suture and the guide line by withdrawing the clamping instrument 1, and leading out the suture line 2 and the guide lines 3 from the small skin incision by using the threading hole 6 on the clamping instrument 1;
S4: fixing the suture line 2 by guiding the suture line 2 by using the guide lines 3, so that both ends of the suture line 2 sequentially penetrate through the Achilles tendon along the guide lines 3; threading the end of suture line 2 on the same side of coil into the coils respectively, so that the suture line 2 pass through the Achilles tendon channel of the guide lines 3 and the Achilles tendon are sutured;
S5: connecting the Achilles tendon by tightening, knotting and fixing the suture line 2 on the broken Achilles tendon.

Before positioning the Achilles tendon, a small skin incision is required at the broken site of the Achilles tendon with a length of 2.0 cm.

While performing the fixation of suture line 2, the end of suture line 2 on the same side of coil is inserted into the coils 4 on the guide lines 3 respectively, where the coils 4 are used to limit the suture line 2. Under the movement of the guide lines 3, one end of the suture line 2 passing through the coil 4 is driven to retreat from outside of the skin incision into the inside of skin incision, so that the suture line 2 penetrates through the Achilles tendon from the same side to the opposite side along the Achilles tendon channel of the guide lines 3, and then is guided out of the skin incision.

As connecting the Achilles tendon, the suture line 2 is tightened to make the two broken Achilles tendon closely contact; after that, the suture line 2 is tied and fixed, and the redundant suture line 2 is cut off.

### Embodiment 2

The application relates to a lead method of suturing acutely ruptured Achilles tendon as shown in FIGS. 3 and 5, which specifically include the following steps:
S1: positioning of the Achilles tendon: inserting a clamping instrument 1 into the fascia sheath of the Achilles tendon through a small skin incision to clamp the Achilles tendon;
S2: threading of the Achilles tendon: using suture needle 7 to make suture line 2 and guide lines 3 penetrate the Achilles tendon and skin from threading hole 6 on clamping instrument 1, where suture line 2 is located far away from Achilles tendon rupture, and guide lines 3 are located between suture line 2 and Achilles tendon rupture;
S3: leading-out of the suture and the guide lines: withdrawing the clamping instrument 1, and leading out the suture line 2 and the guide lines 3 from the small skin incision by using the threading hole 6 on the clamping instrument 1;
S4: fixing of the suture line 2: guiding the suture line 2 by using the guide lines 3, so that both ends of the suture line 2 sequentially penetrate through the Achilles tendon along the guide lines 3; inserting the end of suture line 2 on the opposite side of the coil into the coils respectively, so that the suture line 2 bypasses the Achilles tendon and then passes through the Achilles tendon channel of the guide lines 3 to suture the Achilles tendon;
S5: connecting of the Achilles tendon: tightening, knotting and fixing the suture line 2 on the broken Achilles tendon.

Before positioning the Achilles tendon, a small skin incision is required at the broken end of the Achilles tendon with a length of 2.0 cm.

When the suture line 2 is fixed, the end of the suture line 2 on the opposite side is inserted into the coils 4 on the guide lines 3 respectively, where the coils 4 are used to limit the suture line 2; under the movement of the guide lines 3, one end of the suture line 2 passing through the coil 4 is driven to retreat from the outside of the small skin incision into the inside of the small skin incision, so that the suture line 2 bypasses the rear of the Achilles tendon aponeurosis, then passes through the Achilles tendon along the channel of the guide lines 3 from the opposite side to the same side, and then is guided out of the small skin incision.

As connecting the Achilles tendon, the suture line 2 is tightened to make the two broken Achilles tendon in close contact, then the suture line 2 is tied and fixed, and the redundant suture line 2 is cut off.

In the description of the present application, it should be understood that the terms vertical, horizontal, up, down, front, back, left, right, longitudinal, transverse, top, bottom, inside and outside, etc. indicate an orientation or positional relationship based on the orientation or positional relationship shown in the accompanying drawings and are intended only to facilitate the description of the application; they do not indicate or imply that the device or element referred to must have a particular orientation, or be constructed and operated in a particular orientation, and are therefore not to be construed as limiting the application.

The above-mentioned embodiments only describe the preferred mode of the present application, and do not limit the scope of the present application. Without departing from the design spirit of the present application, all kinds of modifications and improvements made by ordinary technicians in the field to the technical scheme of the present application should fall within the protection scope determined by the claims of the present application.

What is claimed is:

1. A lead method of suturing an acutely ruptured Achilles tendon, comprising:
S1: positioning an Achilles tendon by inserting a clamping instrument into a fascia sheath of the Achilles tendon through a small skin incision to clamp the Achilles tendon;
S2: threading the Achilles tendon by using a suture needle to make a suture line and guide lines penetrate the Achilles tendon and skin from the threading hole on the clamping instrument, wherein the suture line is located far away from an Achilles tendon rupture, and the guide lines are located between the suture line and the Achilles tendon rupture;
S3: leading-out the suture line and the guide lines by withdrawing the clamping instrument, and leading the suture line and the guide lines out of the small skin incision by using the threading hole on the clamping instrument;
S4: fixing the suture line by guiding the suture line with the guide lines, so that both ends of the suture line sequentially penetrate through the Achilles tendon along the guide lines and suture the ruptured Achilles tendon; and
S5: connecting the Achilles tendon by tightening, knotting and fixing the suture line on two broken parts of the Achilles tendon.

2. The lead method according to claim 1, wherein before positioning the Achilles tendon, the small skin incision is made at a broken site of the Achilles tendon, and the small skin incision is between 1.5 cm and 2.5 cm long.

3. The lead method according to claim 1, wherein when the suture line is fixed, the suture line is inserted into coils on the guide lines, and the coils are used to limit the suture line; the coils on the guide lines move with the guide lines and pass through an Achilles tendon channel of the guide lines.

4. The lead method according to claim 3, wherein an end of the suture line on a same side of the coils penetrates into the coils on the guide lines respectively, so that the suture line passes through the Achilles tendon channel of the guide lines.

5. The lead method according to claim 3, wherein an end of the suture line on an opposite side of the coils is inserted into the coils on the guide lines respectively, so that the suture line passes through the Achilles tendon channel of the guide lines after bypassing the Achilles tendon.

6. The lead method according to claim 1, wherein when connecting Achilles tendon, the suture line is tightened to make the two ruptured Achilles tendons in close contact, and a redundant suture line is cut off after the suture line is tied and fixed.

7. A device used in the lead method of suturing an acutely ruptured Achilles tendon according to claim 1, comprising:
a suture line fixedly stitched on a broken Achilles tendon and used to connect the broken Achilles tendon;
guide lines used for guiding the suture line; each guide line being provided with a coil in a middle of the guide lines for limiting the suture line, so as to ensure that the suture line moves along with the guide lines and passes through an Achilles tendon channel of the guide lines;
a clamping instrument used for clamping and fixing the Achilles tendon; front ends of the clamping instrument being provided with threading holes, and the threading hole being used for positioning the suture line and the guide lines; and
suture needles, through which the suture line and the guide lines pass through the Achilles tendon and skin through the threading holes on the clamping instrument.

8. The device according to claim 7, wherein the clamping instrument is clamping anastomosis forceps, and a buckle is arranged at a tail end of the clamping instrument; the buckle is used for fixing an opening and closing angle of the clamping instrument so as to clamp and fix the Achilles tendon.
